(19) United States
(12) Patent Application Publication
(10) Pub. No.: US 2008/0207927 A1
(43) Pub. Date: Aug. 28, 2008

(54) PROCESS FOR THE PRODUCTION OF GLYCEROL ACETALS

(76) Inventors: Setsuo Sato, Sao Jose dos Campos (BR); Alexssander Shigueru Araujo, Sao Jose dos Campos (BR); Miriam Miyano, Sao Jose dos Campos (BR)

Correspondence Address: COGNIS CORPORATION;PATENT DEPARTMENT, 300 BROOKSIDE AVENUE, AMBLER, PA, 19002, US

(21) Appl. No.: 11/862,217

(22) Filed: Sep. 27, 2007

(30) Foreign Application Priority Data
Sep. 28, 2006 (EP) 06020345.2

(51) Int. Cl. C07D 407/04; C07D 319/06; C07D 317/10

(52) U.S. Cl. 549/370; 549/369; 549/448; 549/453

(57) ABSTRACT

A process for the production of glycerol acetals of aldehydes selected from the group consisting of isobutyraldehyde, 2-ethylhexyl aldehyde, furfuryl aldehyde and benzaldehyde, in which glycerol is reacted with one or more of those aldehydes in the absence of organic solvents and in the presence of phosphoric acid as catalyst, the molar ratio of glycerol to aldehyde(s) being between 1:1 to 1:1.2.

### CROSS-REFERENCE TO RELATED APPLICATIONS

This application claims priority under 35 U.S.C. § 119 from European Patent Application No. 06020345.2, filed Sep. 28, 2006, the entire disclosure of which is hereby incorporated by reference.

### BACKGROUND OF THE INVENTION

1. Field of the Invention

This invention relates to an improved process for the production of special glycerol acetals. The process is distinguished by the fact that it does not use a solvent, provides high-purity products and gives excellent yields despite moderate reaction times.

2. Existing Practice

Acetals and ketals of glycerol are known. They are generally produced by reaction of glycerol with aldehydes or ketones in the presence of organic solvents and organometallic catalysts, such as dibutyl tin dichloride, or acidic catalysts, such as p-toluenesulfonic acid.

The problem addressed by the present invention was to provide an improved process for the production of glycerol acetals of isobutyraldehyde, 2-ethylhexyl aldehyde, furfuryl aldehyde and/or benzaldehyde which would not use a solvent, would provide high-purity products and would give excellent yields after moderate reaction times. The products obtained would be colorless without any need for special further purification steps.

The glycerol acetals of isobutyraldehyde, 2-ethylhexyl aldehyde, furfuryl aldehyde and benzaldehyde of particular interest have the following formulae:

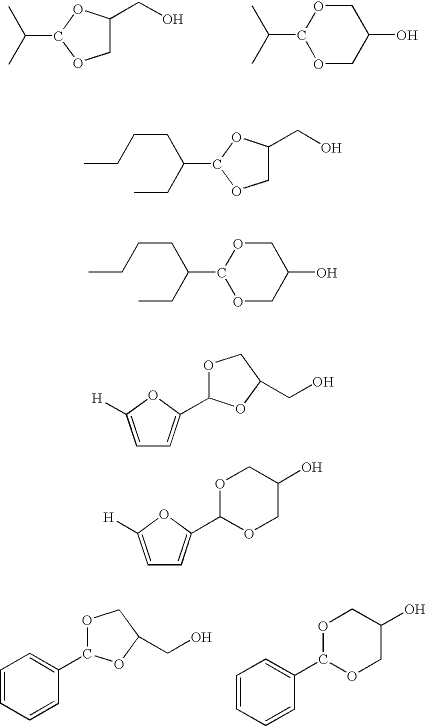

The problem stated above has been solved by the process according to the present invention.

### BRIEF SUMMARY OF THE INVENTION

The essential steps of the process according to the invention are as follows:

1. Glycerol is reacted with one of more aldehydes from the group consisting of isobutyraldehyde, 2-ethylhexyl aldehyde, furfuryl aldehyde and benzaldehyde
2. in the absence of organic solvents and
3. in the presence of phosphoric acid as catalyst,
4. the molar ratio of glycerol to aldehyde(s) being adjusted to a value of 1:1 to 1:1.2 (a ratio of 1:1.1 being particularly preferred).

### DETAILED DESCRIPTION OF THE INVENTION

The present invention relates to a process for the production of glycerol acetals of aldehydes selected from the group consisting of isobutyraldehyde, 2-ethylhexyl aldehyde, furfuryl aldehyde and benzaldehyde, in which glycerol is reacted with one or more of those aldehydes in the absence of organic solvents and in the presence of phosphoric acid as catalyst, the molar ratio of glycerol to aldehyde(s) being adjusted to a value of 1:1 to 1:1.2.

In the interests of clarity, it is pointed out that, where the molar ratio of glycerol to aldehydes is mentioned, the singular form “aldehyde” refers to either isobutyraldehyde or 2-ethylhexyl aldehyde or furfuryl aldehyde or bezaldehyde being used, while the plural form “aldehydes” refers to one or more of those aldehydes being used. Accordingly, the glycerol to aldehyde ratio is always understood to be the molar ratio of glycerol to the totality of the aldehyde(s) present.

In the process according to the invention, the required acetals are obtained in a high yield (at least 95%, based on the quantity of glycerol used). The acetals have a gas-chromatographic purity of at least 95% (percentage area GC) without any need for special purification steps, such as filtration or additional distillation. In addition, the products obtained are colorless. This is not a trivial aspect because tests conducted by applicants have shown that dark-colored products are obtained where p-toluenesulfonic acid, rather than phosphoric acid, is used as the catalyst.

The molar ratio of glycerol to aldehydes is preferably adjusted to a value of ca. 1:1.1.

Glycerol may be used in pure form or with a water content of up to 20% by weight in the process according to the invention. Water-containing glycerols are available, for example, from a biodiesel process.

The process according to the invention is distinguished by short to moderate reaction times. A time of 3 to 6 hours is generally sufficient for a substantially quantitative conversion.

The catalyst used (phosphoric acid) is preferably used in a quantity of 0.1 to 0.8 mol-% (and preferably in a quantity of 0.2 to 0.6 mol-%), based on amount of glycerol used. The reaction is carried out at temperatures of 400 to 150° C. and preferably at temperatures of 70° to 130° C. In one embodiment, the water of reaction is continuously removed from the reaction mixture, preferably using an apparatus of the Dean Stark type.

The reaction is preferably carried out in an inert gas atmosphere, for example, under nitrogen.

In the process according to the invention, the 5- and 6-rings of the particular corresponding acetals (see the above formulae) accumulate in a ratio of ca. 80:20.

The present invention also relates to the use of the glycerol acetals obtainable by the process according to the invention as a solvent, diluent, emollient/plasticizer and wetting agent for various applications, for example, in the fields of cosmetics, textiles and leather, as metalworking lubricants, for agrochemicals and plastics, in the rubber industry, etc. In addition, the glycerol acetals obtainable by the process according to the invention may be used as starting materials for chemical syntheses, more particularly those where the glycerol acetals are reacted with reactants which are capable of reacting with OH groups.

So far as the suitability of the glycerol acetals obtainable by the process according to the invention is concerned, particular emphasis is placed on their suitability as solvents for polymers and resins, more particularly for alkyd and polyester resins, solid polyamides, liquid epoxy resin hardeners, polyurethanes, nitrocellulose, maleic acid-based resins. The glycerol acetals obtainable by the process according to the invention may completely or partly replace traditional solvents.

### EXAMPLES

### Example 1

A 50-liter multipurpose reactor equipped with a mechanical stirrer and distillation head with a water separator and nitrogen inlet was charged under nitrogen at 20° C. with 10195 g (110.8 mol) glycerol, 40.0 g (0.35 mol) phosphoric acid (85% in water) and 9445 g (131 mol) isobutyraldehyde (the main object of the excess was to fill the dead space in the water separator). The temperature was increased to 130° C. over 3 hours. During the reaction, the water of reaction that formed was continuously removed. The reaction was terminated after 2 hours at 130° C. Excess isobutyraldehyde was discharged with a stream of nitrogen, 730 g isobutyraldehyde being recovered. The quantity of water removed amounted to 2200 g. The target product (glycerol acetal of isobutyraldehyde) was obtained in a yield of 16720 g and was characterized as follows:

| | |
|---|---|
| Yield | 97% |
| APHA color | 30 |
| Density (25° C.) g/cm3 | 1.048 |
| Refractive index (20° C.) | 1.447 |
| Freezing point (° C.) | <−50 C. |
| Solubility in water | Soluble |
| Solubility in soybean oil fatty acid | >50% |
| methyl ester | |

### Example 2

A 5000 ml four-necked glass flask equipped with a mechanical stirrer and distillation head with a water separator and nitrogen inlet was charged under nitrogen at 20° C. with 2000 g (21.74 mol) glycerol and 7.25 g (0.06 mol) phosphoric acid (85% in water). The temperature was increased to 110° C. 1724 g (23.91 mol) isobutyraldehyde was then added over a period of 1 hour from a dropping funnel. During the reaction, the water of reaction that formed was continuously removed. The reaction was terminated after a total of 3 hours at 110° C. Excess isobutyraldehyde was discharged with a stream of nitrogen. The target product (glycerol acetal of isobutyraldehyde) was obtained in a yield of 3282 g and was characterized as follows:

| | |
|---|---|
| Yield | 97% |
| APHA color | 5 |
| Density (25° C.) g/cm3 | 1.045 |
| Refractive index (20° C.) | 1.446 |
| Freezing point (° C.) | <−50° C. |
| Solubility in water | Soluble |
| Solubility in soybean oil fatty acid | >50% |
| methyl ester | |

### Example 3

In this Example—in contrast to the preceding Examples—a glycerol-containing residue was used instead of pure glycerol. This residue had been obtained in the production of fatty acid methyl esters by transesterification of native triglyceride oils with methanol in the presence of sodium methanolate as catalyst. The glycerol-containing residue which, besides glycerol, contained water, a little methanol and the sodium methylate catalyst used is referred to hereinafter as crude glycerol. The crude glycerol had a glycerol content of 80% by weight. Because of the sodium methanolate present in the crude glycerol (see below), a comparatively large quantity of phosphoric acid was used because part of the phosphoric acid was first neutralized by the sodium methanolate present.

Acetal formation was carried out as follows:

A 3000 ml four-necked glass flask equipped with a mechanical stirrer and a distillation head with a water separator and nitrogen inlet was charged under nitrogen at 20° C. with 1111 g (9.74 mol) of glycerol in crude glycerol and 127 g (1.1 mol) of phosphoric acid (85% in water). The temperature was increased to 110° C. 764 g (10.6 mol) isobutyraldehyde was then added over a period of 1 hour from a dropping funnel. During the reaction, the water of reaction that formed was continuously removed. The reaction was terminated after a total of 3 hours at 110° C. After cooling to 70° C., 30 g (0.53 mol) potassium hydroxide was added to neutralize the phosphoric acid catalyst. The reaction mixture was then filtered, 1430 g of target product and 200 g of residue being obtained. The target product was then subjected to distillation. A clear end product boiling at 90 to 120° C. under a pressure of 650 mmHg was obtained and was characterized as follows:

| | |
|---|---|
| Yield | 100% |
| APHA color | 50 |
| Gardner color | — |
| Density (25° C.) g/cm3 | 1.049 |
| Refractive index (20° C.) | 1.448 |
| Freezing point (° C.) | −61.2 |
| Solubility in water | Soluble |
| Solubility in soybean oil fatty acid | >50% |
| methyl ester | |

What is claimed is:

1. A process for the production of a glycerol acetal of an aldehyde that has been selected from the group consisting of isobutyraldehyde, 2-ethylhexyl aldehyde, furfuryl aldehyde and benzaldehyde, in which glycerol is reacted with the aldehyde in the absence of organic solvents and in the presence of phosphoric acid as catalyst, the molar ratio of glycerol to aldehyde being adjusted to a value of 1:1 to 1:1.2.

2. A process as claimed in claim 1, characterized in that the glycerol used has a water content of at most 20% by weight.

3. A process as claimed in claim 1, characterized in that the process is carried out at temperatures of 70 to 130° C.

4. A process as claimed in claim 1, characterized in that the phosphoric acid is used in a quantity of 0.1 to 0.8% by volume, based on the volume of glycerol used.

5. A process as claimed in claim 1, characterized in that any water of reaction is continuously removed from the process mixture.

6. A process as claimed in claim 1, characterized in that the process is carried out in an inert gas atmosphere.

7. A solvent for polymers and resins comprising one or more of the glycerol acetals obtained by the process claimed in claim 1.

8. A diluent comprising one or more of the glycerol acetals obtained by the process claimed in claim 1.

9. An emollient comprising one or more of the glycerol acetals obtained by the process claimed in claim 1.

10. A plasticizer comprising one or more of the glycerol acetals obtained by the process claimed in claim 1.

11. A wetting agent comprising one or more of the glycerol acetals obtained by the process claimed in claim 1.

12. A starting material for chemical synthesis where the material is reacted with materials that are capable of reacting with OH groups, where the starting material is one or more of the glycerol acetals obtained by the process claimed in claim 1.
